(19) United States
(12) Patent Application Publication
(10) Pub. No.: US 2018/0193312 A1
(43) Pub. Date: Jul. 12, 2018

(54) NUTRACEUTICAL COMPOSITION AND DOSING REGIMEN

(71) Applicant: ChronoSyn, LLC, Sequim, WA (US)

(72) Inventors: Mark Herbert Swanson, Sequim, WA (US); Paula A Witt-Enderby, Bethel Park, PA (US)

(73) Assignee: ChronoSyn, LLC, Sequim, WA (US)

(21) Appl. No.: 15/658,719

(22) Filed: Jul. 25, 2017

Related U.S. Application Data
(60) Provisional application No. 62/366,775, filed on Jul. 26, 2016.

(51) Int. Cl. A61K 31/4045; A61K 31/593; A61K 31/122; A61K 31/194; A61P 19/10; A61P 15/00; A23L 33/155

(52) U.S. Cl. CPC A61K 31/4045; A61K 31/593; A61K 31/122; A61K 31/194; A61P 19/10; A23V 2200/306; A23L 33/155; A23V 2002/00; A23V 2250/7106; A23V 2250/7144; A61P 15/00

(57) ABSTRACT

The present invention is a nutraceutical formulation, intended for oral administration, comprising co-administered if not co-formulated melatonin together with at least one of strontium, Vitamin D, and Vitamin K or their analogues. The nutraceutical combination or formulation is designed to be taken by mouth within about two hours prior to bedtime, and in fact should not be taken at any other time. The combination therapy gives new and unexpectedly improved results compared to the administration of melatonin alone.

### BACKGROUND OF THE INVENTION

### Field of the Invention

A pharmaceutical/nutraceutical composition effective to prevent or to treat osteoporosis or osteopenia, particularly but not exclusively in post-menopausal women, in need of such treatment, by means of a method of treatment employing a composition containing only nutrients and an endogenous hormone—rather than other pharmaceutical agents of the prior art. The composition and dosing regimen improve bone health, bone density and formation, sleep, weight (changing the progression of stem cells from forming fat, into forming bone), diabetes risk, general well-being and sexual desire and function in post-menopausal women.

### Description of Related Art

A well known indication predominantly (but not exclusively) in women, particularly post-menopausal women, is osteoporosis (and osteopenia), that is, either the treatment of it or, ideally, the prevention of it. At this writing there are myriad approved pharmaceutical treatments for osteoporosis, having well described and documented benefits—and, like all pharmaceuticals, inevitable unwanted side effects. Brand name drugs well known for treatment of osteoporosis include, without limitation, Boniva, Actonel, Binosto, Fosamax, Reclast, Evista, Forteo, and Prolia. The mechanism of action of these drugs work in various ways, including interfering with bone loss, increasing bone strength, and so forth.

Notwithstanding the widespread availability of existing pharmaceuticals that are FDA-approved to address (to prevent or to treat) osteoporosis, the prevention or treatment osteopenia is typically prescribed to people with a prior history of vertebral or hip fracture and/or have a high risk of future fracture. There is also a sizeable segment of the osteoporosis-osteopenic population in which these pharmaceuticals have either failed, been refused or discontinued by the patient, or for various reasons are not a suitable option. The most common reason for refusal and discontinuing is an unacceptably high incidence of side effects, often serious. For example, bisphosphonates are the most widely prescribed class of osteoporosis drugs, yet gastrointestinal pain and irritation occur in almost half the people. Less common, but more serious adverse effects are osteonecrosis of the jaw and atypical femoral fractures. Not surprisingly, the compliance to oral bisphosphonates is suboptimal, and the adherence and persistence is poor.

Bone loss is defined medically as osteopenia, and osteoporosis most often afflicts postmenopausal women although it is not in itself gender specifical Osteoporosis often asymptomatic until a fracture occurs, or multiple asymptomatic fractures can also occur, and patients often have an aversion to taking prescription drugs in the absence of symptoms or prior fracture. This has resulted in an unacceptable “treatment void” contributing significantly to a reduction in the quality of life (QOL), bone fracture, debilitating chronic pain and disability, and earlier death attributed to osteoporosis. In the case of osteopenia an early, non-invasive, and effective treatment would completely reverse these risks. Pharmaceutical treatment options are typically postponed for up to 10 years or more, when in the absence of previous fracture, until a diagnosis of osteoporosis is reached. Not addressed is the fact that almost 10 times more fractures occur in osteopenic patients than with osteoporosis. Clearly, were it available, having the option of a safer and exceptionally well tolerated, research-based nutraceutical combination, proven effective for increasing bone health in the osteopenic population, is urgently needed. Such a nutraceutical would ideally be equally suitable for application to osteoporosis, to offer both as an alternative or complementary option to pharmaceuticals. Nutraceutical intervention is especially important today, given the fact that despite many years of routine recommendations for taking supplemental calcium and vitamin D for bone health—as a universally adopted nutritional standard for osteopenia and osteoporosis—the current evidence is insufficient to assess the balance of the benefits and harms of daily supplementation in this way. Many nutritional supplements containing calcium and vitamin D meet FDA compliance for a label claim stating “reduces the risk of osteoporosis.” However, because calcium and vitamin D in systematic reviews of clinical studies show only marginal and minimal efficacy, this claim is largely an illusionary one. To foreshadow the invention described later in this specification, it is interesting that the present invention is highly efficacious against osteoporosis and osteopenia, without any necessary inclusion of any calcium at all, whereas the vast majority of prior art nutritional supplements that contain calcium are largely or completely ineffective against osteoporosis or osteopenia.

Menopausal transition, although a natural physiological phenomenon, is often accompanied by the debilitating consequences of bone loss such as osteoporosis and related fractures. Currently, about 9 million U.S. adults are suffering from osteoporosis and another 50 million osteopenic people are under great threats of developing osteoporosis in the future. These numbers are predicted to be increased up to 11.9 million for osteoporosis and 64.3 million for osteopenia by 2030. Postmenopausal women comprise a significant portion in this population, posing high risk of having one or more fractures (approximately 40%) in their lifetimes. In fact, the projected annual total fracture costs across all fracture types for the US women is more than 18 billion by 2025, which is more than that of myocardial infarction, stroke and breast cancer. In this way, the “silent” disease osteoporosis is creating a loud impact in terms of morbidity, mortality and greater economic burden in the life of postmenopausal women and worsening their health related quality of life. The large multiethnic Study of Women's Health Across the Nation (SWAN) explains the changes in health related quality of life over the menopausal transition by symptoms related to menopause and/or aging, such as disrupted sleeping, depressed mood, perceived stress and stressful life events.

Most of the existing pharmaceutical bone loss therapies, of the prior art, emphasize only treating bone loss and for reducing fractures, without adequately focusing enough on new bone formation. Anabolic agents reduce fracture incidence by improving bone qualities besides increasing bone mass. Bone anabolism is a critical therapeutic step for maintaining normal bone integrity and microarchitecture. Again, to foreshadow, the present invention has the multiple actions of treating bone loss while acting as an anabolic treatment by stimulating new bone growth and also modulating proteins involved in energy metabolism, while also preventing recurring fractures. Other unwanted side effects of certain prior art therapies include, without limitation, a) osteonecrosis of the jaw and atypical femoral shaft fractures with biphosphonates, b) osteonecrosis of the jaw, eczema and cellulitis with denosumab, c) deep venous thrombosis and hot flashes with raloxifene, and d) risk of osteosarcoma with Teriperatide (the only anabolic therapy currently available in the prior art). As always, unwanted side effects and risks inevitably compromise therapy adherence and compliance among postmenopausal women, resulting in a known 37% increase in the risk of hospitalization and associated cost of medical services. All of these necessitate the development of a safe and effective treatment that will not only stop bone loss but also enrich new bone growth and improve overall quality of life n the post-menopausal women population.

### SUMMARY OF THE INVENTION

In order to meet this need and achieve all the above mentioned goals and desirable outcomes, the present invention is a nutraceutical formulation, intended for oral administration, comprising co-administered if not co-formulated melatonin, strontium (preferably citrate), Vitamin D3, and Vitamin K2 (MK7). The nutraceutical combination or formulation is designed to be taken by mouth within about 30 minutes—two hours prior to bedtime, and in fact should not be taken at any other time. Dosing is most preferably 5 mg melatonin, 450 mg strontium (citrate), 2000 IU vitamin D₃ and 60 μg vitamin K₂, with possible flexibility in dosing ranges of 0.1-20 mg, more preferably 2-10 mg, melatonin; 0.1 mg to 2 g, more preferably 200-700 mg, strontium; 0.1-10,000 IU, more preferably 1000 to 3000 IU, vitamin D₃; and 0.1-300 μg, more preferably 15-100 μg, vitamin K₂. In addition to improved results in preventing and treating osteoporosis and osteopenia, a new and surprisingly improved result has been shown in that post-menopausal women who take the present nutraceutical composition every day within two hours' prior to bedtime, for a period of at least six consecutive months of consecutive daily doses, can expect a natural increase and return of sexual libido, desire and function compared to post-menopausal women who do not take the present nutraceutical supplement, while at the same time the present combined administration or co-formulation shifted human mesenchymal cells from adipogenesis to osteoblastogenesis, meaning that stem cells that could have turned into fat cells—became new bone cells in the bone, instead. The well-being implications of the present invention, for post-menopausal women, are significant and profound.

### DETAILED DESCRIPTION OF THE INVENTION

The invention is a specific method of nutraceutical supplementation in which prevention, reduction or treatment of osteopenia or osteoporosis or osteopenia is indicated, by administering a synergistic combination of melatonin with active supplement agents within two hours' time prior to bedtime. The dosing amounts set forth in the previous paragraph may be adjusted, according to the skill of the art, to accommodate patients whose body weights vary significantly from an average body weight. The active agents of the present invention, as well as their compounding and oral dosage form preparation generally, are already known in the art, and the present invention is directed to the improvement of co-administration of melatonin, strontium, Vitamin K2, and Vitamin D in the improved therapies disclosed herein. Suitable types and salts of strontium may be selected from the group consisting of, but not limited to, strontium citrate, strontium carbonate, strontium malate, strontium gluconate, strontium ascorbate, strontium glycinate, strontium chloride, strontium hydroxyapatite, strontium threonate or other strontium salts and combinations thereof. As described immediately above, the present nutraceutical combination or formulation is designed to be taken by mouth within about two hours prior to bedtime, and in fact should not be taken at any other time because of melatonin's nighttime circadium activity.

Prior to the present invention, studies have shown that sufficient sleep is important to the promotion of healthy sexual desire and genital response, as well as the likelihood of engaging in partnered sexual activity. These relationships were independent of daytime affect and fatigue. Unique to the present invention, in addition to improved results in preventing and treating osteoporosis and osteopenia, a new and surprisingly improved result, with the invention described herein, has been shown in that post-menopausal women who take the present nutraceutical composition every day within two hours' prior to bedtime, for a period of at least six consecutive months of consecutive daily doses, can expect a natural increase and return of sexual desire and function, compared to post-menopausal women who do not take the present nutraceutical supplement. This wellness improvement is a new and surprising benefit and, prior to the present invention, would clearly have been medically counterintuitive, as follows. Melatonin in general is so ubiquitously associated with bedtime and sleep enhancement that its ability to cooperate with other nutrients to improve sexual function would have seemed, prior to the present invention, to have suggested, “Will this make me too sleepy for sexual activity?” The inventors have discovered that the opposite is true. When post-menopausal women take the present nutraceutical as prescribed, daily and for at least six consecutive months, the favorable improvement in sexual desire increases, and without interfering or reducing the improvements in sleep quality attributed to melatonin, if taken as monotherapy. Daytime sleepiness did not increase as a result of the present invention, which is further indication of the promotion of sleep quality with an accompanied increase in sexual desire. The present invention describes for the first time the increased sexual desire (without need to wait until cessation of sexual activity for intake or other dosing) and the wellness effect of good overall sleeping and sleep schedules (going to bed on time, around 10 pm or so, and waking up on time, around 7 am or so) without napping or at least without napping too frequently. The clinical study results that led the inventors to the above conclusions, not only as to improved sexual function and desire in post-menopausal women on a chronobiology basis, but also as to the measurable improvements in their osteoblast (bone forming cells) formation, gave rise to the illustrative and descriptive memorable marketing characterization, Osteoblast Your Libido!, which according to the confirmed data in the present invention turns out to be literally true, as well as difficult to ignore.

Although the above described constituents are the most preferred, some substitution and combinations are possible with the inventive formulations with co-administrations with melatonin, in that any form of strontium or strontium salt or combination thereof may be used in the present invention as the strontium component. Likewise, any form of Vitamin D₃, Vitamin D₂ or Vitamin D analogs or combinations thereof may be used in the present invention as the Vitamin D component. Likewise, any form of Vitamin K₂ (MK7), Vitamin K₂ (MK4) or Vitamin K₁ or Vitamin K₃ or combinations thereof may be substituted in the present regimen for the Vitamin K constituent. Likewise, the suitable nutraceutical combinations can also consist of melatonin and strontium; melatonin, strontium and Vitamin D; or melatonin, strontium and Vitamin K. Likewise, the addition of any other nutraceutical ingredient without limitation, such as to include vitamins, minerals, amino acids, plant extracts, antioxidants, hormones, polyphenols, flavonoids, etc., may be used in the present invention with any of the suitable nutraceutical combinations. However, melatonin per se is a constant of the present invention; there is no ingredient that can substitute for melatonin, except for melatonin analogues and agonists.

Although the preferred route of administration of the present invention is oral, it is also possible to formulate and to administer the present constituents, for co-administration or as a co-formulation, via the routes of administration including transdermal, dermal using a topical lotion, gel or cream, transmucosal including vaginal, sublingual, buccal, pessary, etc., or parenteral. Adjusting dosing for these alternate-to-oral routes of administration is known in the art, and takes into consideration the ability of the constituent to bypass the hepatic first pass effect while also compensating for reduced delivery through the epidermis compared to mucosa, and so forth.

However, to simplify compliance, the combined active agents may desirably be compounded together in an oral dosage form intended for administration at or near (within two hours of) bedtime. The present nutraceutical should be taken at least two hours after food, milk, milk products or calcium supplements, preferably at bedtime, and is intended for long-term use. Patients should also receive calcium or vitamin D supplements if they are not getting enough from their diet, after taking into consideration any vitamin D administered with the present supplement. A typical oral dosage form could contain 450 mg strontium (citrate), 2000 IU vitamin D3, 60 mcg Vitamin K2, and 5 mg of melatonin, together with customary pharmaceutically acceptable excipients and diluents, in a form typically intended for oral administration such as a tablet(s), a capsule(s), caplet(s), caplique(s), lozenge(s), sachet(s), stick pack(s), fast-dissolve wafer(s) or strip(s), or piece(s) of chewing gum, without limitation. The oral dosage form may also be in a liquid solution, containing one or more of the above-described active agents or other similar active agents described below; if the active agents are in separate solutions the combination therapy may be implemented by administering one or more solutions to the patient at the same time. The active agents may also be administered individually or in any combination and delivery method in the nighttime time frame described herein. The combined active agents, including suitable combinations, may also be used as constituents or additives in food supplements including but not limited to nutrition bars, nutrition wafers, nutrition powders, nutritional beverage mixes and prepared drinks—typically containing but not limited to one or more of the following ingredients in any combination: protein, carbohydrate, fats and oils, fatty acids, vitamins and minerals, fiber, prebiotics, probiotics, herbals, plant extracts, amino acids, antioxidants, polyphenols, alkaloids, sweeteners, or other nutraceutical constituents. Optimally, when the combined active ingredients include additional nutritional components—such as protein, carbohydrate, etc.—ideally the traditional nutritional constituents are taken at least 3-hours prior to the bedtime dose of the invention to minimize nighttime digestion and a subsequent rise in the postprandial glucose and insulin responses affecting glucose tolerance. Taking the combined active ingredients, if also containing calcium from dairy or other source of calcium, should also be at least 3-hours prior to the bedtime dose containing melatonin plus the additional active(s), to encourage strontium absorption and bioavailability. Although most usually the combined active agents are given with minimized if any additional calories due to the optimal late day (within two hours of bedtime) administration of at least the melatonin component, it is also general wisdom that not eating late at night and ceasing daily caloric intake well before bedtime is beneficial to overall health promotion and weight control.

Furthermore, the medically optimal way to administer the combinational therapy is to administer strontium, vitamin D, vitamin K and melatonin orally in a single or multiple capsules, tablets, dissolvable beverage powder preparations, such as powder in packets, or as a single dose liquid-type “shot,” even though other routes of administration are acceptable. Delivery methods may also include specific preparations—involving the active ingredients, excipients, or encapsulation, tableting or other routes-of-delivery materials—to include nano and micro particle, micro-encapsulation, liposomes, phytosomes, emulsions, oil-surfactants, fatty acid, colloidal dispersions, cyclodextrins, polyethylene glycol, controlled release, sustain-release, delayed-release, timed-release, bi-phasic release, gastro-retentive, gastric acid-resistant, enzyme coupling, compounded, prebiotic and probiotic combinations, amino acid, and any other manufacturing method that is intended to enhance absorption, bioavailabilty, digestive comfort, gut receptors and microbiome diversity, and therapeutic effectiveness, including desirable off-target and any additional therapeutic or quality-of-life (QOL) enhancing applications.

A good “kit” type formulation for the present invention is in combination with a daytime calcium supplement, vitamin D supplement, or any other vitamin, mineral and nutraceutical combination, in any dosage and combination, with the present formulation or co-administration at night (that is within two hours of bedtime), to include any additional vitamin, mineral (except calcium), nutraceutical, or combinations thereof. The inventors envision a dynamic and continual 24/7 therapy period with this combination that “slows bone loss during the day and stimulates new bone formation at night.”

Assertions made in this specification are corroborated in published data (Maria, S., Swanson, M. H., Enderby, L. T., D'Amico, F., Enderby, B., Samsonraj, R. M., Dudakovic, A., van Wijnen, A. J., Witt-Enderby, P. A., “Melatonin-micronutrients Osteopenia Treatment Study (MOTS): a translational study assessing melatonin, strontium (citrate), vitamin D3 and vitamin K2 (MK7) on bone density, bone marker turnover and health related quality of life in postmenopausal osteopenic women following a one-year double-blind RCT and on osteoblast-osteoclast co-cultures,” Aging, 9 (1): 256-285, 2017), collected during a formal clinical study. Comprehensive treatment of the study results will therefore be available from public sources when examination of this patent specification begins and/or can be considered in corroborated form. Having said that, however, key conclusions from both the clinical trial and the MenQOL (Menopause-Specific Quality of Life questionnaire) sexual domain scores are summarized as follows. The combination therapy discussed above significantly increased lumbar spine bone mineral density (BMD) by 4.3% and left femoral neck BMD by 2.2%, with a possible trend towards increase in hip BMD from baseline after one year in post-menopausal osteopenic women. This is comparable to bisphosphonates. As a consequence, the 10-year vertebral fracture risk probability was decreased by 6.48%, as compared to 10.8% increase in placebo. The inventive therapy significantly increased bone formation marker P1NP, while not increasing bone resorption marker Ctx. This was shown to be due to an increase in osteoblast formation, a decrease in osteoclast formation and a decrease in osteoblastic metabolic markers (i.e., PPAR gamma and GLUT4) known to be involved in diabetes and targets of diabetic pharmacotherapies. The present inventive therapy also showed positive effects on inflammatory status, height, weight, and lean body mass and also lessened the sexual symptoms of menopause, increased sexual libido and desire, and showed improvements with respect to sleep quality, gastrointestinal upset and general aches and pains. In addition, the present formulation or co-administration surprisingly shifted human mesenchymal cells from adipogenesis to osteoblastogenesis (fat to bone). With respect to restoration of sexual function and interest, we relied on the results of the MenQOL-Intervention survey conducted with members of the clinical trial. MenQOL vasomotor, physical, and psychosocial domain scores did not change significantly with the present treatment. However, after twelve months of treatment with the present invention, improvements in sexual domain scores occurred opposite to what was observed for placebo—which was completely unexpected because no change in the MenQOL scores for sexual domain changed, in a related prior study. The prior study was a clinical trial investigation of melatonin alone, in which administration of 3 mg melatonin for six months did show an improvement in MenQOL scores as to physical domain but no improvement as to sexual domain. The improvement in sexual domain occurred concurrently, in the clinical trial population of the present invention, to the measurable improvement in bone density attributable to the co-administration of the herein disclosed constituents, with melatonin per se as a constant of the present invention.

1. A nutraceutical method of treating multiple indications relating to osteopenia and osteoporosis, comprising administering one time per day, within two hours of bedtime, at least one unit dose of melatonin combined with at least one dose of one of strontium, Vitamin D or Vitamin K or their analogues in an appropriate vehicle or dosage form for a human patient in whom such treatment is indicated.

2. The nutraceutical method of claim 1, wherein said strontium is a strontium salt, said Vitamin D is Vitamin D3, and said Vitamin K is Vitamin K2, and further wherein all four of melatonin, Vitamin D3, Vitamin K2 and a strontium salt are present in said nutraceutical.

3. The nutraceutical method of claim 1, wherein said unit doses are separate or combined in a dosage form suitable for oral administration.

4. The nutraceutical method of claim 1, wherein said unit doses are separate or combined in a dosage form suitable for parenteral, transmucosal, transdermal, or topical delivery.

5. The nutraceutical method of claim 1, wherein said unit doses are packaged in a kit, in which a separate dosage form containing calcium or calcium with and at least one form of Vitamin D is also present, prior to administration.

6. The nutraceutical method of claim 1, wherein said administration occurs daily for a minimum of six consecutive months.

7. The nutraceutical method of claim 1, wherein said administration occurs daily for a minimum of one year.

8. The nutraceutical method of claim 1 wherein, as a result of the administration, bone density, bone formation, in said post-menopausal woman increases and post-menopausal sexual function and interest also increase, compared to post-menopausal women who are not treated according to the method of claim 1.

9. A nutraceutical composition containing 0.1-20 mg melatonin, 0.1 mg to 3 g strontium (citrate), 0.1-10,000 IU vitamin D3 and 0.1-300 μg vitamin K2.

10. (canceled)

11. A nutraceutical composition containing 5 mg melatonin, 450 mg strontium (citrate), 2000 IU vitamin D3 and 60 μg vitamin K2.

12. A nutraceutical composition according to claim 11 wherein at least one pharmaceutically acceptable excipient is present in said composition.

13. A nutraceutical composition wherein a kit contains at least two separate dosage components forms for administration, a daytime kit component containing calcium, calcium and Vitamin D, or any other vitamin, mineral, herb, plant extract, fatty acid, or nutraceutical in any dosage and combination and a composition according to claim 9 for administration within two hours of bed time.

14. A nutraceutical composition according to claim 9 with the addition of any other vitamin, mineral, herb, plant extract, fatty acid, amino acid, or nutraceutical or food nutrient in any dosage and combination, with the exception of calcium.

15. The nutraceutical method of claim 1 wherein, as a result of the administration, metabolic function, in said post-menopausal woman improves compared to post-menopausal women who are not treated according to the method of claim 1.

16. The nutraceutical method of claim 1 wherein, as a result of the administration, diabetes risk, in said post-menopausal woman decreases compared to post-menopausal women who are not treated according to the method of claim 1.

17. The nutraceutical composition in kit form according to claim 13, wherein all compositions are formulated for oral administration.

18. A method of treating a patient at risk for low bone mass, bone loss, osteopenia and osteoporosis by administering, in unit dosage form, one or more dosages of the nutraceutical set forth in claim 1 on a daily basis.

19. The method according to claim 18 wherein said daily treatment continues for a period of at least six months.
